(19) United States
(12) Patent Application Publication
(10) Pub. No.: US 2011/0270118 A1
(43) Pub. Date: Nov. 3, 2011

(54) MEASURING AND DISPLAYING THE PROPAGATION VELOCITY OF UTERINE ACTION POTENTIALS TO DETERMINE THE ONSET OF LABOR

(75) Inventors: Robert Garfield, Goodyear, AZ (US); William L. Maner, Phoenix, AZ (US); Rainer Fink, College Station, TX (US); Jack N. McCrary, Houston, TX (US)

(73) Assignee: REPRODUCTIVE RESEARCH TECHNOLOGIES, LP, Houston, TX (US)

(21) Appl. No.: 13/021,037

(22) Filed: Feb. 4, 2011

Related U.S. Application Data
(60) Provisional application No. 61/301,271, filed on Feb. 4, 2010.

(51) Int. Cl. A61B 5/0488

(52) U.S. Cl. 600/546

(57) ABSTRACT

A method and system to examine and measure the propagation velocity of electrical activity in pregnant patients, labor and non-labor patients at term and preterm, and non-pregnant patients, thereby providing valuable information regarding the state of a patient's uterus. The method can include obtaining uterine EMG signals from a series electrodes, processing the raw uterine EMG signal in a signal processing module and assessing the temporal interval between adjacent electrodes. The propagation velocity can then be estimated by averaging the time required for the uterine EMG signal to traverse a distance between adjacent electrodes.

### BACKGROUND

The diagnosis of labor (term and preterm) is a significant challenge faced by obstetricians. Preterm labor is the pathological state most frequently associated with this dilemma. Moreover, term labor often requires adjuvant therapy to halt or augment labor. However, there is no currently available method to objectively and accurately diagnose when the uterus is prepared to labor either preterm or term. Since there is spontaneous uterine activity throughout pregnancy, it is generally not possible to distinguish between physiological uterine activity and preterm labor. The state of the cervix is commonly used as a predictor of preterm birth. However, the softening of the cervix, as well as the appearance of uterine contractions often occur relatively late in preterm labor.

The uterus is generally inactive throughout pregnancy to maintain a tranquil environment for the growing fetus. At the end of pregnancy, however, the uterus normally begins to contract forcefully in a phasic manner (labor) to expel the fetus and other products of conceptions. Abnormally, the uterus may either begin to contract and labor prior to term (preterm labor) or fail to contract at term. In most cases the clinician is faced with the decision to either inhibit labor or stimulate it depending on the circumstances. However, the clinician typically has only subjective methods (state of cervix or number of contractions but not force of contraction) on which to base a decision.

The uterus is now known to pass through a series of steps prior to and during labor to prepare the muscle to contract in a coordinated, synchronous and therefore forceful manner. These steps include the development of gap junctions (low electrical resistance contacts), receptors and other events between and on the muscle cells that allow the uterus to contract as a syncytium and react to contractile agents. Contractions of the uterus are dependent upon electrical activity, such as action potentials propagated through the uterus. Therefore, the presence of gap junctions is an important component of this process. When the muscle cells pass through this state they become electrically and metabolically coupled, thereby allowing the uterus to contract forcefully and frequently. However, at present, the obstetrician or gynecologist has no objective method to evaluate this process. As can be appreciated, the clinical judgment as to treatment would be greatly enhanced by systems and methods which could define the state of the patient's uterus.

What is needed, therefore, is a method and system to examine and measure the propagation velocity of electrical activity in labor and non-labor patients at term and preterm, thereby providing valuable information regarding the state of a patient's uterus.

### SUMMARY

Embodiments of the disclosure provide a method of measuring propagation velocity of uterine contractions. The method may include applying a series of electrodes to a maternal abdomen of a patient, obtaining analog uterine EMG signals representative of a uterine contraction from the series of electrodes, and processing the analog uterine EMG signals in a signal processing module to obtain digital EMG signals. The method may further include determining a temporal interval for the digital EMG signals between the series of electrodes, and calculating a propagation velocity of the uterine contraction from the determined temporal interval and a distance between electrodes. The calculated propagation velocity may then be displayed to a user via a variety of formats.

Embodiments of the disclosure may further provide another method of measuring propagation velocity of uterine contractions. The other method may include receiving an EMG signal(s) at a first electrode pair, receiving the EMG signal(s) at a second electrode pair, determining a temporal interval of the EMG signal(s) between the first and second pair of electrodes, and calculating a propagation velocity of the EMG signal(s). The labor status can then be determined based on the calculated propagation velocity. The propagation velocity or labor status may then be displayed to a user.

### BRIEF DESCRIPTION OF THE DRAWINGS

The disclosure is best understood from the following detailed description when read with the accompanying Figures. It is emphasized that, in accordance with the standard practice in the industry, various features are not drawn to scale. In fact, the dimensions of the various features may be arbitrarily increased or reduced for clarity of discussion.

FIG. 1 illustrates a schematic of the uterine electrical activity analyzer system according to one or more embodiments of the disclosure.

FIG. 2 illustrates EMG signals captured from at least two electrodes, compared with a tocodynamometer signal, and providing the time lapse between adjacent electrodes.

FIG. 3 illustrates a bar chart indicating the resulting propagation velocities for term labor/non-labor and preterm labor/non-uterine.

FIG. 4 illustrates a plot graph indicating propagation velocity in the uterus of test patients at or near delivery.

FIG. 5 illustrates a receiver operating characteristics curve indicating the sensitivity versus the specificity of the systems described herein when applied to patients within seven days of delivery.

FIG. 6 illustrates a receiver operating characteristics curve indicating the sensitivity versus the specificity of the systems described herein when applied to patients within twenty-four hours of delivery.

### DETAILED DESCRIPTION

It is to be understood that the following disclosure describes several exemplary embodiments for implementing different features, structures, or functions of the invention. Exemplary embodiments of components, arrangements, and configurations are described below to simplify the present disclosure, however, these exemplary embodiments are provided merely as examples and are not intended to limit the scope of the invention. Additionally, the present disclosure may repeat reference numerals and/or letters in the various exemplary embodiments and across the Figures provided herein. This repetition is for the purpose of simplicity and clarity and does not in itself dictate a relationship between the various exemplary embodiments and/or configurations discussed in the various Figures. Moreover, the formation of a first feature over or on a second feature in the description that follows may include embodiments in which the first and second features are formed in direct contact, and may also include embodiments in which additional features may be formed interposing the first and second features, such that the first and second features may not be in direct contact. Finally, the exemplary embodiments presented below may be combined in any combination of ways, i.e., any element from one exemplary embodiment may be used in any other exemplary embodiment, without departing from the scope of the disclosure.

Additionally, certain terms are used throughout the following description and claims to refer to particular components. As one skilled in the art will appreciate, various entities may refer to the same component by different names, and as such, the naming convention for the elements described herein is not intended to limit the scope of the invention, unless otherwise specifically defined herein. Further, the naming convention used herein is not intended to distinguish between components that differ in name but not function. Further, in the following discussion and in the claims, the terms “including” and “comprising” are used in an open-ended fashion, and thus should be interpreted to mean “including, but not limited to.” All numerical values in this disclosure may be exact or approximate values unless otherwise specifically stated. Accordingly, various embodiments of the disclosure may deviate from the numbers, values, and ranges disclosed herein without departing from the intended scope. Furthermore, as it is used in the claims or specification, the term “or” is intended to encompass both exclusive and inclusive cases, i.e., “A or B” is intended to be synonymous with “at least one of A and B,” unless otherwise expressly specified herein.

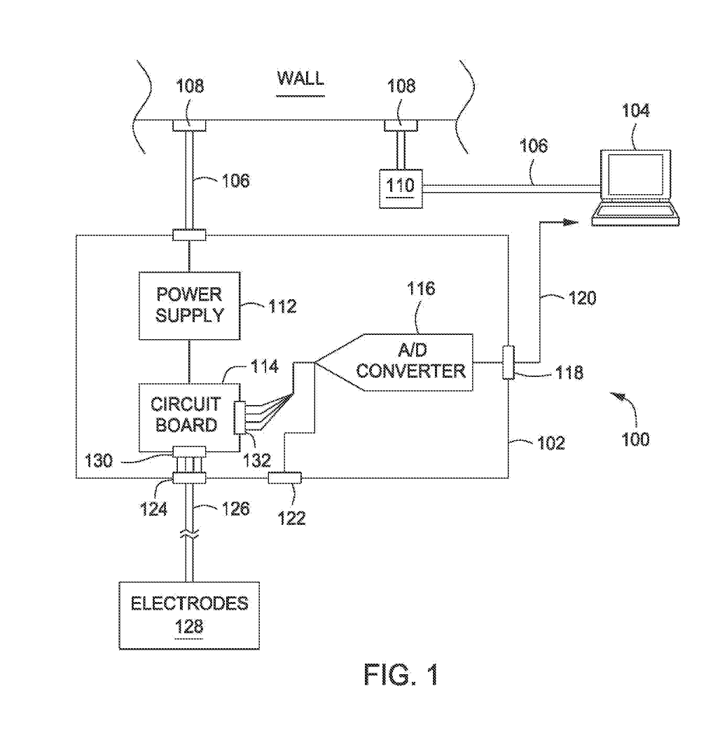

Referring to FIG. 1, illustrated is a system 100 for acquiring and processing uterine electromyography or electromyogram (“EMG”) signals. As known by those skilled in the art, EMG can also be known as or substantially similar to electrohistography or electrohistograms (“EHG”). Consequently, the acquisition and processing of EHG signals is also contemplated herein, without departing from the scope of the present disclosure. A uterine EMG signal is the functional equivalent to a uterine activity signal created by a tocodynamometer (“toco”) or Intrauterine Pressure Catheter (“IUPC”), but can be a great deal more precise. The global muscle contractions of the uterus triggered by an action potential can be seen externally as an EMG signal. When electrodes are placed on the abdomen, they measure the global muscle firing of uterine contractions, thereby resulting in a “raw” uterine EMG signal.

The system 100 may include a signal processing module 102 communicably coupled to and/or integral to a computer 104. The signal processing module 102 and the computer 104 may each include hardware, however, the computer 104 may include software for executing machine-readable instructions to produce a desired result. In at least one embodiment, the software may include an executable software program created in LABVIEW® or other similar software products. The hardware may include at least processor-capable platforms, such as client-machines (also known as personal computers or servers) and hand-held processing devices (such as mobile phones, personal digital assistants (PDAs), or personal computing devices (PLDs), for example). Further, hardware may include any physical device that is capable of storing machine-readable instructions, such as memory or other data storage devices, and executing those instructions (e.g., via a processor). Other forms of hardware include hardware sub-systems, including transfer devices such as modems, modem cards, ports, and port cards. In short, the computer 104 may include any other micro processing device, as is known in the art. The computer 104 may include a monitor for displaying processed uterine EMG signals, labor status, or propagation velocity for evaluation. The computer 104 may also be communicably coupled to a printer (not shown) for providing a printed report of such results.

In an exemplary embodiment, the computer 104 may include, without limitation, a desktop computer, laptop computer, or a mobile computing device. Moreover, the computer 104 may include a CPU and memory (not shown), and may also include an operating system (“OS”) that controls the operation of the computer 104. The OS may be a MICROSOFT® Windows OS, but in other embodiments, the OS may be any kind of operating system, including without limitation any version of the LINUX® OS, any version of the UNIX® OS, or any other conventional OS as is known in the art.

Both the signal processing module 102 and the computer 104 may be powered via a medical-grade power cord 106 that may be connected to any typical wall outlet 108 conveying 120 volts of power. As can be appreciated, the system 100 may also be configured to operate on varying voltage systems present in foreign countries. For the computer 104, however, the power cord 106 may include an interim, medical-grade power brick 110 configured to reduce or eliminate leakage current originating at the wall outlet 108 that may potentially dissipate through the internal circuitry of the system 100 or a patient.

The signal processing module 102 may house a power supply module 112, a circuit board module 114, and an analog to digital (“A/D”) converter 116. The power supply module 112 may be configured to supply power for the signal processing module 102. In particular, the power supply module 112 may receive 120V-60 Hz power from the wall outlet 108 and convert that into a 12 volt direct current to be supplied to the circuit board module 114. In alternative embodiments, the power supply module 112 may be configured to receive varying types of power, for example, DC current from a battery or power available in foreign countries.

In an embodiment, the circuit board 114 may be an electronic circuit configured to receive, amplify, and filter the incoming uterine signals. In particular, a series of high-pass and low-pass filters may first be configured to amplify and filter the incoming uterine EMG signals to frequencies broadly located between about 0.2 Hz to about 2 Hz, the typical frequency of uterine EMG activity found in humans (e.g. for the embodiment used for labor status determination). The EMG signals may further be filtered and amplified with computer software forming part of the system 100 to frequencies ranging from about 0.3 Hz to about 1.0 Hz, thereby obtaining a more precise signal representative of uterine activity (e.g. for the embodiment used for labor status determination). During the filtration process, software manipulation of the data may include removing any motion artifacts, or stray signals resulting from patient movement or someone contacting the electrodes or leads and thereby causing a spike in signal activity. To accomplish this, the software may be programmed with a uterine EMG threshold that automatically disregards registered signals that exceed that limit. Alternative software data manipulation may include altering the gain of the signal, and calculating the root mean square of the data to obtain a signal representative of uterine activity, as commonly seen in the toco and IUPC. Furthermore, it is also contemplated to acquire a signal substantially equivalent to the root mean square by taking a low-pass filter frequency (e.g., 0.01 Hz). Such an equivalent signal will also be similar to a signal as commonly seen in the toco and IUPC.

The ND converter 116 may digitize the incoming analog uterine signals into a viewable digital signal transmittable to the computer 104 for display. Specifically, the ND converter 116 may be communicably coupled to an external USB port 118 located on the body of the signal processing module 102. A double-ended USB connection cable 120 may be utilized to communicably couple the USB port 118 to the computer 104. However, in other embodiments the USB port 118 may be replaced with a wireless adapter and signal transmitter to wirelessly transmit the processed uterine data directly to a receiver located on the computer 104.

The signal processing module 102 may also include one or more toco, IUPC, fetal heart rate, maternal heart rate, or other communication port(s) 122 through which physicians may be able to acquire and process uterine signals via a tocodynamometer or IUPC, as is already well-known in the art. For example, through the communication port 122, physicians may be able to track a toco signal, IUPC signal, maternal heart rate, and/or fetal heart rate, and also acquire intrauterine pressures via an IUPC or chronicle uterine activity via a toco or other instruments. The analog signals sent to the communication port 122 may be directed to the ND converter 116 to be digitized and subsequently displayed through the computer 104.

Similarly, the signal processing module 102 may further include an EMG communication port 124 which may be communicably coupled to one or more pairs of electrodes 128 and a patient ground electrode via an EMG channel 126. Through the electrodes 128, physicians may acquire and process raw uterine EMG signals. Specifically, the electrodes 128 may be configured to measure the differential muscle potential across the area between the two pairs of electrodes 128 and reference that potential to patient ground. In at least one embodiment, the processed uterine EMG signal(s) may provide the propagation velocity of electrical activity in labor and non-labor patients at term and preterm.

Once the muscle potential is acquired, the raw uterine EMG signal(s) may then be routed to an input 130 for processing within the circuit board 114. After processing within the circuit board 114, the processed uterine EMG signal(s) may be directed out of the circuit board 114, through an output 132, and to the ND converter 116 where the analog uterine EMG signal(s) may be subsequently digitized for display on the computer 104. Although only one EMG channel 126 is illustrated in FIG. 1, the disclosure fully contemplates using multiple EMG channels 126—each EMG channel 126 being communicably coupled to a pair of electrodes 128. For example, in at least one embodiment there are two or more pairs of electrodes 128 used to measure the propagation velocity of uterine contractions.

The functionality and structure of the system 100, and particularly the signal processing module 102, is further described in co-pending U.S patent application Ser. No. 12/696,936, filed on Jan. 29, 2010, and entitled “SYSTEM AND METHOD FOR ACQUIRING AND DISPLAYING UTERINE EMG SIGNALS,” the contents of which are incorporated herein by reference in their entirety, to the extent that they are not inconsistent with the present disclosure.

According to several embodiments of the present disclosure, the propagation velocity of uterine electrical signals can be measured using the system 100 as generally described herein. During this non-invasive procedure, uterine EMG signals may yield valuable information about the electrical coupling of myometrial cells required for term and preterm labor. Such measurements can then be displayed and analyzed to accurately distinguish between true and false labor at term and/or preterm, among other types of uterine contractions. As can be appreciated, the ability to distinguish between true and false labor can be highly advantageous since a considerable amount of resources can be spent in “waiting” to verify true/false labor. As used herein, the phrase “term” can mean greater than thirty-seven weeks of gestation, and the phrase “preterm” can mean less than thirty-seven weeks of gestation (i.e., premature labor).

The foregoing discussion can be further described, without being bound by any theory, with reference to the following non-limiting example, which is used to verify and demonstrate that the system 100 described herein may be used to accurately measure the propagation velocity of uterine electrical signals, which in this example, was to determine the status of labor. A study on electrical propagation during term and preterm labor was undertaken involving ninety-eight (98) pregnant women whose maternal ages ranged from 18 to 43 years. At the beginning of the study, twenty-eight (28) women were at or near term, and seventy (70) women were considered preterm. Twenty-two (22) of the term patients delivered within 24 hours from undertaking the uterine EMG measurement (i.e., “term labor”), while six (6) did not deliver within 24 hours (i.e., “term non-labor”). The preterm patients were admitted with a diagnosis of threatened preterm labor. Eighteen (18) of the preterm patients delivered within 7 days from undertaking the uterine EMG measurement (i.e., “preterm labor”), while fifty-two (52) did not deliver within 7 days (i.e., “preterm non-labor”).

A four-electrode 128 arrangement was used to acquire the uterine EMG contractile activity of each patient. For comparison purposes, tocodynamometry was simultaneously undertaken using a commercially-available toco instrument strapped to the abdomen of the patient. The electrode 128 arrangement was symmetric about the navel of each patient, with the vertical and horizontal axes parallel to the patient vertical and horizontal axes, respectively, and with center-to-center distances between adjacent electrodes set at 5.0 to 5.5 cm apart. As can be appreciated, however, embodiments of the present disclosure contemplate variations in electrode 128 spacing (any range between about 0.5 cm and 32 cm; e.g. ranges between any one or more of 0.5, 1, 2, 3, 4, 5, 6, 7, 8, 9, 10, 12, 14, 16, 18, 20, 24, 28, and 32 cm), distancing (any range between about 0.5 cm and 32 cm; e.g. ranges between any one or more of 0.5, 1, 2, 3, 4, 5, 6, 7, 8, 9, 10, 12, 14, 16, 18, 20, 24, 28, and 32 cm), number (e.g. 2, 3, 4, 5, 6, 7, 8, 9, . . . n), and general arrangement (e.g. symmetrical, nonsymmetrical, etc.) without departing from the scope thereof.

The electrodes 128 were placed on each patient for at least ten minutes prior to initiating signal capture, and grounding was accomplished by placing an electrode laterally on the patient's hip (i.e., to electrically connect the patient to ground electrical potential and thus eliminate interfering signals). Uterine EMG was then measured for approximately 30 minutes using the system 100 as generally described herein. In the embodiments disclosed herein, differential, bipolar electrode 128 pairs were used. Thus, the propagation velocity was assessed by finding a temporal interval at adjacent electrode 128 pairs, rather than at individual electrodes 128. In other embodiments, however, different types of electrodes 128 could be implemented without departing from the scope of the disclosure.

Each patient was asked to remain as still as possible and in a supine position so as to avoid disturbing any of the probes and wires for the EMG and/or toco. Analog EMG signals were then acquired and digitally filtered to yield a final band-pass of about 0.34 to about 1.00 Hz, and sampled at 100 Hz. In at least one embodiment, the digital filtering is undertaken to exclude noise components apparent during the analysis, such as motion, respiration, and cardiac signals.

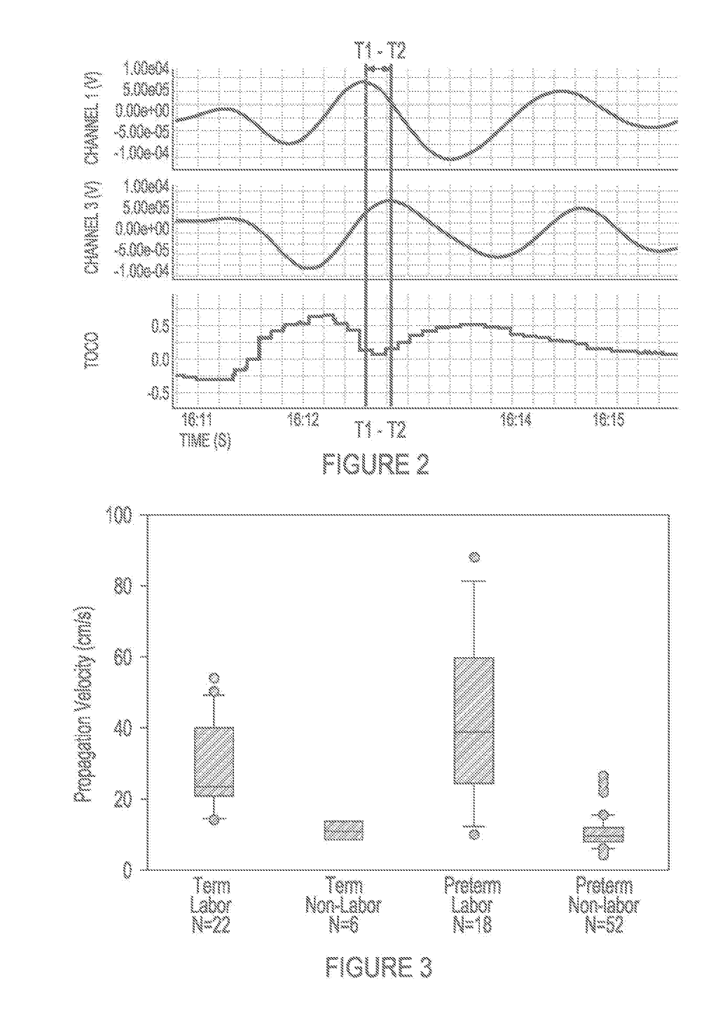

Referring now to FIG. 2, propagation velocity was then determined from the temporal interval between EMG signal arrivals originating from adjacent electrodes 128 (e.g., Channel 1 and Channel 3) and their respective order of appearance. The average time required for the propagating signal to traverse the distance between adjacent electrodes 128 was then assessed by looking at all of the time differences in corresponding action potential peaks at adjacent electrode 128 pairs for each burst of action potentials. The average of absolute values was then taken of all time differences for bursts for the patient's uterine EMG recording. As illustrated in FIG. 2, a propagating myometrial wave impinges/maxes out upon electrode pair 1 (Channel 1) at time T1, and shortly thereafter impinges/maxes out upon electrode pair 2 (Channel 3) at T2.

Referring now to FIG. 3, analysis of variance was then implemented to compare the propagation velocity in term labor, term non-labor, preterm labor, and preterm non-labor groups (P21 0.05 significant). As is apparent from the bar chart depicted in FIG. 3, the uterine propagation velocity tends to be significantly higher in patients during labor at term and preterm as compared to non-labor states. For example, propagation velocity was significantly higher (P<0.001) in labor at term (mean 31.25±14.91 cm/s) and preterm (mean 47.20±31.24 cm/s) compared with non-labor patients at term (mean 11.31±2.89 cm/s) and preterm (mean 11.27±5.61 cm/s). In other words, patients at term gestation and measuring a propagation velocity of about 31.25±14.91 cm/s, are almost certain to be in true labor. However, patients at term gestation and measuring a propagation velocity of about 11.31±2.89 cm/s are likely experiencing false labor symptoms. Likewise, preterm gestation patients measuring propagation velocities of about 47.20±31.24 cm/s are likely experiencing true labor, while those measuring propagation velocities of about 11.27±5.61 cm/s are likely experiencing false labor. The differences between labor and non-labor, where the propagation velocities are drastically different, can be considered an unexpected result that can, in at least one embodiment, accurately indicate real and false labor.

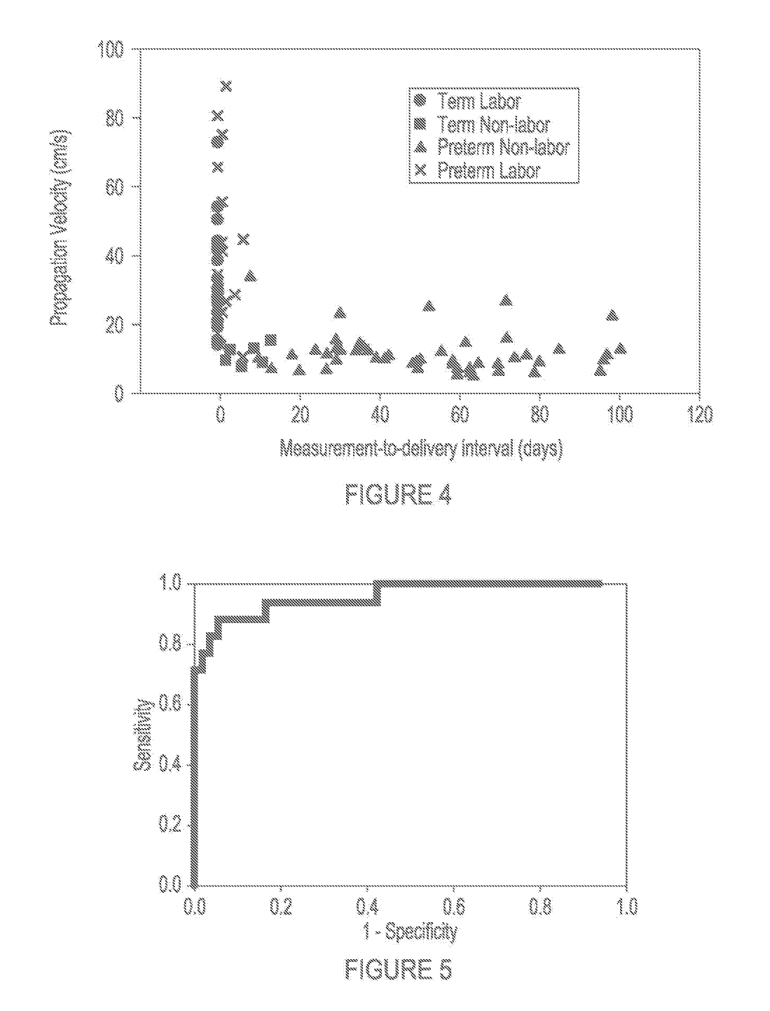

Referring now to FIG. 4, the illustrated chart indicates that uterine propagation velocity generally increases as the measurement-to-delivery interval decreases in both term and preterm patients. This increase in propagation velocity generally occurs about 24 hours prior to delivery in term delivering patients (N=22), and about 7 days prior to delivery in preterm delivering patients (N=18).

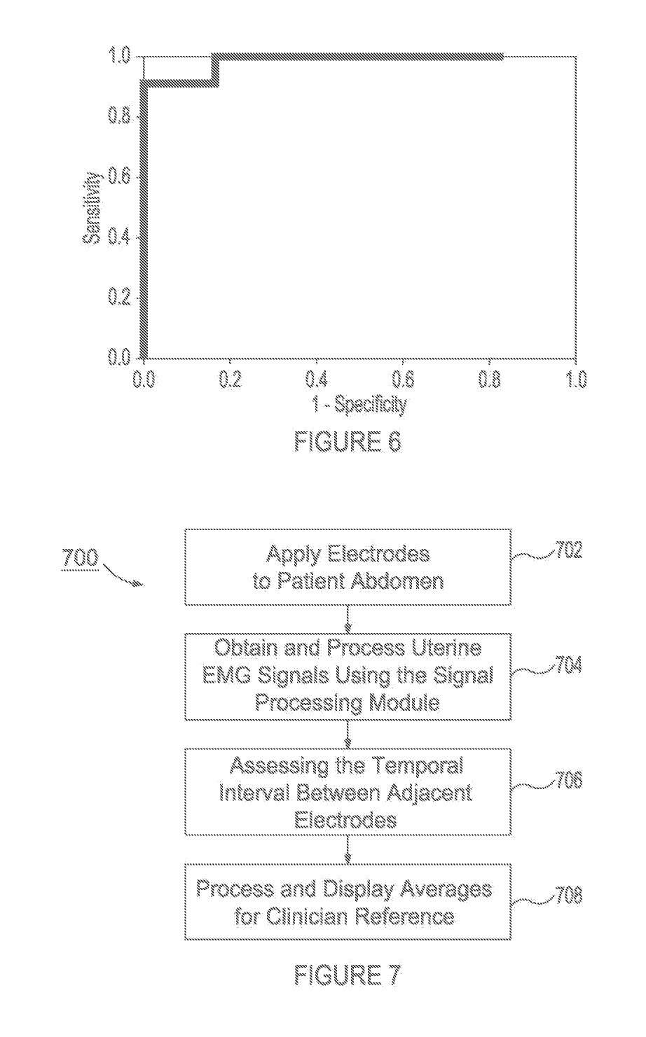

Referring to FIGS. 5 and 6, receiver operating characteristic analysis was used to assess the diagnostic accuracy of measuring the propagation velocity in predicting delivery within 7 days in preterm patients N=70 (see FIG. 5) and within 24 hours in term patients N=28 (see FIG. 6). Both FIGS. 5 and 6 provide the sensitivity vs. specificity related to the system 100 to indicate how accurate the method of delivery prediction was for each respective time period. In FIG. 5, an end point of 7 or fewer days to delivery was used to generate the curve. The resulting area under the curve was about 0.96, indicating that the system 100 accurately registered true labor contractions 96% of the time. Results from FIG. 5 further indicated a 100% positive predicted value and a negative predicted value of 91%. According to the results, the best cut-off propagation velocity for determining true labor is about 26.60 cm/s for preterm patients.

In FIG. 6, an end point of 24 or fewer hours to delivery was used to generate the curve. The resulting area under the curve was about 0.98, indicating that the system 100 accurately registered true labor contractions 98% of the time. Results from FIG. 6 further indicated a 96% positive predicted value and a negative predicted value of 100%. Furthermore, according to the results in FIG. 6, the best cut-off propagation velocity for determining true labor is about 13.19 cm/s for term patients.

Referring to FIG. 7, a schematic is depicted indicating an exemplary method 700 of measuring the propagation velocity of uterine contractions. The method 700 can include applying a series of electrodes 128 to a maternal abdomen of a patient, as at block 702. As explained above, the general arrangement of the electrodes 128 can be configured to match the vertical and horizontal axes of the patient and centered around the navel. In other embodiments, however, the general arrangement of electrodes 128 can be square, rectangular, or a variation thereof (i.e., tilted on an angle with respect to vertical). Furthermore, the series of electrodes 128 can be four or more electrodes coupled or otherwise attached to the maternal abdomen.

Uterine EMG signals can then be obtained from each pair of electrodes 128 and processed in the signal processing module 102 to obtain digital EMG signal(s), as at block 704. To calculate the propagation velocity, the temporal interval between adjacent electrodes 128 can be assessed, as at block 706. As explained above, the temporal interval can include the time required for the uterine EMG signal(s) to traverse the distance between adjacent electrodes 128 (e.g., peak to peak measurement), as shown in FIG. 2 herein. Thus, the distance from center-to-center of each electrode 128 can be measured and taken into account for purposes of calculating the temporal interval and the relative velocity (e.g., Velocity=Distance×Time) of the signals. The averaging results can then be processed and displayed for reference by a gynecologist or clinician, as at block 408. As part of the processing, the results are processed in a computer having software for executing machine-readable instructions to obtain a signal representative of uterine activity.

In one or more embodiments, the system 100 can also be configured to determine propagation directionality. For example, with respect to the general arrangement of the electrodes 128, the electrodes 128 could be adapted to compare the number of EMG signals propagating from the fundus towards the cervix and vice-versa and thereby establish a general direction of propagation. In other embodiments, more than 4 electrodes can be used to accomplish this.

In yet another embodiment, the propagation velocity in any direction can be determined through the use of the cross correlation function in discrete signal processing and paired EMG burst activity. In practice, a single channel of EMG burst activity can be matched to a second channel of EMG burst activity to determine the time differential between the two signals. With a known distance between the two EMG electrodes, a propagation velocity can be determined from the time shift that resulted in the highest cross correlation value. It is disclosed that a propagation velocity could be of interest in any direction in the muscle as well as circumferentially around the uterus, requiring the placement of electrodes in areas of the body other than the center of the stomach.

In still additional embodiments, the methods and apparatus disclosed herein can also be employed to measure other types of uterine contractions such as for the evaluation and determination of dysmenorrhea (e.g. menstrual pain), fertility and implantation, postpartum tonic contraction, the failure of postpartum tonic (or uterine atony) contraction (tetanic), resulting in postpartum hemorrhage, other uterine contractions or the lack thereof, and uterine contraction disorders. However, these and other types of uterine contractions may require different frequency band filtering to achieve an optimal output.

The foregoing has outlined features of several embodiments so that those skilled in the art may better understand the detailed description that follows. Those skilled in the art should appreciate that they may readily use the disclosure as a basis for designing or modifying other processes and structures for carrying out the same purposes and/or achieving the same advantages of the embodiments introduced herein. Those skilled in the art should also realize that such equivalent constructions do not depart from the spirit and scope of the disclosure, and that they may make various changes, substitutions and alterations herein without departing from the spirit and scope of the disclosure.

We claim:

1. A method of measuring propagation velocity of uterine contractions, comprising:
obtaining at least two analog uterine EMG signals representative of a uterine contraction from a series of electrodes;
processing said at least two analog uterine EMG signals in a signal processing module to obtain at least two digital EMG signals;
determining a temporal interval for said at least two digital EMG signals between said series of electrodes;
calculating a propagation velocity of one or more uterine contractions from said determined temporal interval and a distance between said two or more of said series of electrodes; and
displaying said calculated propagation velocity.

2. The method of claim 1, wherein said calculated propagation velocity is used to identify, monitor, diagnose, and/or treat one or more of the following:
labor status;
dysmenorrhea;
fertility and implantation;
postpartum tonic contraction; and
postpartum tetanic contraction.

3. The method of claim 1, wherein said series of electrodes comprises differential bipolar electrodes.

4. The method of claim 1, wherein said series of electrodes comprises two or more pairs of electrodes.

5. The method of claim 4, wherein said two or more pairs of electrodes are arranged symmetrically about a navel of a patient and having vertical and horizontal axes of said electrodes substantially parallel to vertical and horizontal axes of said patient

6. The method of claim 5, wherein said two or more pairs of electrodes are arranged with center-to-center distances between adjacent electrodes set at about 0.5 to about 32.0 cm apart.

7. The method of claim 1, wherein calculating said propagation velocity of said one or more uterine contractions comprises averaging a plurality of calculated velocities.

8. The method of claim 7, wherein calculating said propagation velocity of said one or more uterine contractions further comprises taking an average of absolute values of all time differences for action potential bursts.

9. The method of claim 1, additionally comprising a cross correlation function, said cross correlation function is applied to a first action potential burst and a second action potential burst and said temporal interval is the temporal interval with the highest cross correlation value.

10. The method of claim 1, wherein processing said analog uterine EMG signal in a signal processing module comprises:
amplifying said analog EMG signal;
filtering said analog EMG signal to a frequency band between about 0.2 Hz to about 2.0 Hz to obtain an amplified and filtered analog signal; and
transmitting said amplified and filtered analog signal to an analog to digital conversion module to convert said amplified and filtered analog signal into said digital EMG signal.

11. The method of claim 10, further comprising filtering and amplifying said digital EMG signal to a frequency band between about 0.34 Hz and about 1.0 Hz.

12. The method of claim 10, wherein processing said digital EMG signal in said signal processing module further comprises:
removing motion artifacts from said digital EMG signal; and
determining the root mean square of said digital EMG signal.

13. The method of claim 1, wherein said propagation velocity is displayed on a computer communicably coupled to said signal processing module.

14. A method of measuring propagation velocity of uterine contractions, comprising:
receiving at least two EMG signals at a first electrode pair and a second electrode pair;
determining a temporal interval of said at least two EMG signals between said first electrode pair and said second electrode pair;
calculating a propagation velocity of one or more uterine contractions;
determining a labor status of a patient based on said calculated propagation velocity; and
displaying said propagation velocity and/or labor status.

15. The method of claim 14, additionally comprising a cross correlation function, said cross correlation function applied to a first action potential burst received from said first electrode pair and a second action potential burst received from said second electrode pair and said temporal interval is the temporal interval with the highest cross correlation value.

16. The method of claim 14, wherein said at least two EMG signals are received from at least two pairs of electrodes.

17. The method of claim 14, wherein at least said first electrode pair and said second electrode pair are applied to a maternal abdomen of a patient.

18. The method of claim 17, wherein at least said first electrode pair and said second electrode pair are applied generally symmetrically about a navel of said patient.

19. The method of claim 14, further comprising processing said at least two EMG signals with a signal processing module.

20. The method of claim 19, wherein said signal processing module is communicably coupled to said first electrode pair and said second electrode pair and configured to filter and amplify said EMG signal to a frequency band between about 0.2 Hz to about 2.0 Hz.

21. The method of claim 20, further comprising filtering and amplifying said digital EMG signal to a frequency band between about 0.34 Hz and about 1.0 Hz.

22. The method of claim 14, wherein said labor status is true labor when said propagation velocity is above a limit, and false labor when said propagation velocity is below said limit.

23. The method of claim 14, wherein said propagation velocity or labor status is displayed via a monitor or printed report.
